(19) United States
(12) Patent Application Publication
(10) Pub. No.: US 2026/0226452 A1
(43) Pub. Date: Aug. 6, 2026

(54) METHODS AND APPARATUS FOR EXTRACTING OR SEPARATING NUCLEIC ACIDS FROM BIOLOGICAL SAMPLES

(71) Applicant: Inso Biosciences, Inc., Ithaca, NY (US)

(72) Inventors: Adam Bisogni, Ithaca, NY (US); Irem Bastuzel, Ithaca, NY (US); Harvey C. Tian, Ithaca, NY (US); Mohamed Zakary Rashed, Ithaca, NY (US)

(21) Appl. No.: 19/297,313

(22) Filed: Aug. 12, 2025

Related U.S. Application Data
(60) Provisional application No. 63/683,026, filed on Aug. 14, 2024.
(60) Provisional application No. 63/710,977, filed on Oct. 23, 2024.

(51) Int. Cl. C12N 15/10; C12Q 1/6806

(52) U.S. Cl. CPC C12N 15/1013; C12Q 1/6806

(57) ABSTRACT

The invention provides methods and apparatuses for extracting or separating nucleic acids from biological samples by (i) flowing an aqueous fluid containing a cell through a channel of a microfluidic device, wherein the cell gets lysed and releases, into aqueous solution in the aqueous fluid, at least one DNA molecule of at least one hundred kbp in length, (ii) capturing the DNA molecule on a capture array in the channel, and (iii) changing flow conditions of the aqueous fluid to wash the DNA from the capture array and into a collection reservoir without introducing any fragmentation enzyme or protocol, thereby maintaining the DNA molecule in constant aqueous solution from lysis of the cell to collection in reservoir, and to thus provide the DNA molecule (at least 100 kbp in length) in aqueous solution in the collection reservoir.

### BACKGROUND OF THE INVENTION

Genomic analysis is important in research and medicine in applications ranging from cancer diagnosis to understanding tissue development. Microfluidic devices have been explored for these cell studies, as such devices have the potential to handle small sample and reagent volumes using engineered microstructures. Specifically, efforts have been made to extract DNA from single cell to large cell populations by trapping and lysing cells in a microfluidic device to release DNA strands that are then trapped in micropillar arrays on the device, followed by release of the DNA from the device by restriction endonuclease digestion under continuous flow for off-chip collection. See Benitez, 2014, Microfluidic extraction, stretching and analysis of human chromosomal DNA from single cells, Lap Chip 12(22): 4848-4854, incorporated by reference.

It is understood that it may be difficult to recover long molecules out of such a device due to breakage of strands caused by both the restriction endonuclease digestion and shearing associated with the flow conditions. See Agrawal, 2020, Microfluidic long DNA sample preparation from cells, Lab Chip 19(2): 281-290, incorporated by reference.

### SUMMARY OF THE INVENTION

The invention provides methods and apparatuses for extracting or separating nucleic acids from biological samples under conditions that allow intact, high molecular weight DNA to be collected for further study without any requirement to cleave or fragment the DNA. Methods of the invention provide intact genomic DNA molecules that can be hundreds of thousands of base-pairs (kilo base-pairs, or kbp) and even longer in length. Notably, methods of the invention keep the DNA molecules in aqueous solution throughout the separation from cellular debris and isolation into a collection vessel. DNA isolation operates by flowing a cell or cells, in an aqueous liquid, into a channel that includes cell-capture features and a DNA capture array. The cell gets held at the cell-capture features where the cell is lysed, e.g., by flowing lysis reagents through the channel. DNA released from the lysed cell flows through the channel and is captured, or entangled, on the DNA capture array. The aqueous liquid may be continuously flowed through the channel, which may wash away cellular debris. The DNA capture array may be provided as an array of pillars or obstacles across the channel, and when fluorescent DNA dyes are used, photomicrographs show that the DNA is entangled on the array of obstacles.

The invention exploits the surprising insight that changing flow conditions, such as by increasing pressure or flow rate, can wash the DNA off of the DNA capture array and into a collection vessel without requiring the use of any restriction enzymes or any other enzyme or protocol to break or fragment the DNA. Without being bound by any particular mechanism, it may be that a high pressure wash induces some deformation of the physical geometry of the microfluidic channel or DNA capture array, or adds enough energy via the aqueous liquid, to release or wash the DNA off the capture array. Notable benefits of DNA separated and isolated by methods of the disclosure include that the DNA is maintained in aqueous solution throughout the process, from cell lysis to collection in a vessel (e.g., such as a microcentrifuge tube), and also that DNA so collected is very high molecular weight, never having been exposed to fragmentation conditions such as cleavage enzymes or a shearing protocol. In fact, DNA isolated by methods of the disclosure exhibit notable bands on gels at positions corresponding to DNA molecule lengths above 300 kilo base-pairs (kbp) and even as high as above 700 kbp.

Methods of the invention are useful for the isolation of nucleic acids such as DNA, RNA, plasmids, or other vectors from microorganisms, viruses and metazoan organisms and notably for the isolation of genomic DNA from e.g., plants and animals, particularly vertebrates such as mammals, including specifically from human cells. One or more cells are loaded into a channel within a microfluidic chip and lysed, allowing genomic DNA to be released from the cell but entangled on a DNA capture array within the channel.

The disclosure provides methods to “blast” the DNA of off the capture array and off of the chip without using enzymes. In other words, fluid flow or shear force is used to “snap” or “release” the DNA from the obstacles or pillars of the capture array. Methods of the invention provide several benefits including that the process is fast (not requiring any incubation time), the process requires very little in terms of specialized reagents (e.g., enzymes, special buffers), and the process provide intact, high molecular weight DNA (hundreds of kbp in length).

In certain aspects, the invention provides methods of extracting or separating nucleic acids from a biological sample. Methods include introducing a sample containing a cell into a channel of a microfluidic device; capturing the cell on one or more cell-capture features disposed within the channel; and lysing the captured cell to release DNA from the cell. The DNA is flowed through the channel to a capture array within the channel, where the method further includes capturing the DNA on the capture array and washing the DNA into a collection reservoir by flowing a fluid through the channel under conditions that remove the DNA from the capture array thereby collecting, in the collection reservoir, at least one DNA molecule at least one hundred kilobase-pairs in length that was released from the cell. Preferably, the washing step is performed without introducing any restriction enzyme into the microfluidic device.

In certain embodiments, the conditions that remove the DNA from the capture array include an increase in pressure or volume of the fluid flowing through the channel. In some embodiments, the increase in pressure deforms at least a portion of the channel or the capture array to create or increase spacing through which the DNA is removed from the capture array (the microfluidic device may be made of polymer or plastic such as polydimethylsiloxane (PDMS) bonded to glass or thermos plastic elastomers (TPE) bonded to a hard plastic or another layer of TPE. It may be found that the PDMS or TPE deforms slightly under pressure, enough to introduce or expand a gap through which DNA molecules are released). The capture array may comprise an array of pillars or obstacles extending from a first wall of the channel and terminating at ends that sit against a second wall, opposed to the first wall. In such embodiments, the conditions that remove the DNA from the capture array may include an increase in pressure of the fluid that deforms the microfluidic device (e.g., deforms a polymer or plastic material) to move the first wall away from the second wall to introduce or expand a gap between the second wall and the ends of the pillars or obstacles. The DNA may also be removed through other means of physical agitation to the microfluidic device such as tapping the device or scraping along the channel length.

As results presented herein show, after the washing step, the collection vessel may collect a plurality of DNA molecules each greater than one hundred kilobase-pairs that were released from the cell by the lysing step. For example, preferably after the washing step, the collection vessel collects a plurality of genomic DNA molecules each greater than five hundred kilobase-pairs that were released from the cell by the lysing step. Other smaller fragments may be among those, but the collected DNA includes the molecules that are at least 500 kbp.

It may be that the conditions that remove the DNA from the capture array include an increase in flow rate of the aqueous fluid, such that the increase in the flow rate washes the DNA off of the capture array.

Other embodiments or mechanisms are within the scope of the invention. For example, the conditions that remove the DNA from the capture array may include changing a magnetic field around the microfluidic device to decrease an obstruction presented by the capture array. In some embodiments, the capture array comprises an array of magnetically responsive structures that change orientation in response to the changing of the magnetic field. In other embodiments, the capture array is provided by functionalized magnetic DNA capture beads held in place within the channel by a magnetic field, and the conditions that remove the DNA from the capture array include changing or removing magnetic field (so that the magnetic beads are not being held in place within the channel) and washing the DNA capture beads out of the microfluidic device.

In some embodiments, the capture array comprises an array of solid structures on which the DNA gets captured and the conditions that remove the DNA from the capture array may include retracting (e.g., capture array provided by pillars pushed through holes in a floor of the channel), dissolving (e.g., soluble, but poorly-soluble pillars), or melting (e.g., wax pillars) the solid structures to free the captured DNA. The capture array may be provided by an array of proteins on which the DNA gets captured and the conditions that remove the DNA from the capture array may include introducing a protease or a reducing agent that degrades the proteins. In other related embodiments, the capture array may be provided as pillars or obstacles extending from a first wall of the channel and the conditions that remove the DNA from the capture array may include removing, opening, or lifting away a second wall, opposed to the first wall, to allow the fluid to freely wash the DNA out of the microfluidic device (e.g., opening up the microfluidic device). In certain embodiments, the capture array is provided as an array of obstacles that include a silica resin on which the DNA gets captured and the conditions that remove the DNA from the capture array include washing the capture array with a low salt solution that decreases affinity of the DNA for the silica resin.

In related aspects, the invention provides methods of extracting or separating high molecular weight DNA from a biological sample by (i) flowing an aqueous fluid containing a cell through a channel of a microfluidic device, wherein the cell gets lysed and releases, into aqueous solution in the aqueous fluid, at least one DNA molecule of at least one hundred kbp in length, (ii) capturing the DNA molecule on a capture array in the channel, and (iii) changing flow conditions of the aqueous fluid to wash the DNA from the capture array and into a collection reservoir, thereby maintaining the DNA molecule in constant aqueous solution from lysis of the cell to collection in reservoir, and to thus provide the DNA molecule (at least 100 kbp in length) in aqueous solution in the collection reservoir. The changing flow conditions may include increasing pressure of the aqueous fluid to deform at least a portion of the channel or the capture array to thereby create or increase spacing through which the DNA molecule escapes the capture array. Preferably, the DNA molecule is not exposed to any exogenous endonuclease during progress of the method from lysis of the cell to collection in reservoir. Methods may include collecting numerous genomic DNA molecules, each at least 500 kbp in length, in the collection reservoir, wherein the genomic DNA molecules constitute a substantial portion of a genome of the cell.

Aspects of the invention provide methods of preparing nucleic acid for sequencing. Methods include introducing a sample containing a cell into a channel of a microfluidic device; capturing the cell at a cell capture structure disposed within the channel; lysing the captured cell to release DNA; capturing the DNA at a capture site within the channel; and either (i) flowing a transposome complex to the capture site and, by the action of the transposome, attaching transposase adaptors to segments of the DNA to yield adaptor-linked comprising transposase-adaptors linked to segments of the DNA [tagmentation approach], or (ii) fragmenting the DNA to yield fragments and ligating sequencing adaptors to the fragments to yield adaptor-ligated fragments [ligation approach] (wherein the ligating step may be performed on the microfluidic chip or off-chip, after adjusting flow conditions to wash the fragments from the chip). In certain embodiments of tagmentation approaches, sequencing adaptors are subsequently attached to the adaptor-linked fragments to yield sequencing substrates. In some embodiments of the tagmentation approaches, the transposase adaptors may be sequencing adaptors (e.g., Y-adaptors with a double stranded portion and first and second single stranded ends with a motor protein attached the first single stranded portion). In ligation approaches, methods may include ligating Y-adaptors (optionally with a motor protein) to the fragments to yield adaptor-ligated fragments. Depending on platform, any suitable sequencing adaptors may be used in the various approaches or embodiments including, for example, Y-adaptors (with no motor protein) such as those used on next-generation sequencing instruments sold by Illumina. The ligating step may be performed within a reaction volume on the microfluidic device downstream of the capture site. The methods may include flowing the adaptor-ligated fragments to a sequencing flow cell. The methods may include attaching the microfluidic device and the sequencing flow cell to an instrument that holds the capture site in fluidic communication with an interior volume of the sequencing flow cell. The methods may include flowing library preparation reagents that include the transposome complex to the capture site via a channel that intersects with a DNA flow channel extending from the cell capture structure to the capture site. In some embodiments, the cell capture site comprises a first plurality of structures in a channel configured to trap the cell and wherein the capture site includes a second plurality of structures, sized and/or spaced smaller than the first, configured to entangle and capture genomic DNA.

### BRIEF DESCRIPTION OF DRAWINGS

FIG. 1 diagrams steps of a method of extracting or separating nucleic acids.

FIG. 2 shows an apparatus for extracting or separating nucleic acids from a sample.

FIG. 3 shows a capture array.

FIG. 4 shows a result of increasing pressure.

FIG. 5 shows a gel with DNA that was extracted and isolated using methods herein.

FIG. 6 shows DNA yield.

FIG. 7 is a bar graph showing purity of DNA.

FIG. 8 shows a capture array that includes magnetically-responsive bars.

FIG. 9 shows the device with the magnetically-responsive bars.

FIG. 10 is a photograph of an apparatus of the disclosure.

FIG. 11 shows a capture array sealed with a film.

FIG. 12 shows a capture array with the film unsealed.

FIG. 13 is a photograph of a microfluidic device.

### DETAILED DESCRIPTION OF THE INVENTION

The invention provides methods and apparatuses for extracting or separating nucleic acids from biological samples by (i) flowing an aqueous fluid containing a cell or cells through a channel of a microfluidic device, wherein the cell gets lysed and releases, into aqueous solution in the aqueous fluid, at least one DNA molecule of at least one hundred kbp in length, (ii) capturing the DNA molecule on a capture array in the channel, and (iii) changing or providing flow conditions of the aqueous fluid to wash the DNA from the capture array and into a collection reservoir, thereby maintaining the DNA molecule in constant aqueous solution from lysis of the cell to collection in reservoir, and to thus provide the DNA molecule (at least 100 kbp in length) in aqueous solution in the collection reservoir.

Methods are applicable to any living cell and particularly to prokaryotes, preferably metazoans such as plants and animals. While the nucleic acid that is isolated may be RNA or DNA, and may be plasmids, vectors, organelle DNA, etc., of viral, bacterial, plant, yeast, or animal, preferred embodiments are suited for the extraction or separation of organismal genomic DNA such as from plants, animals, or Eukaryotic microorganisms such as yeast and fungi.

Methods provided herein may be used to “blast” DNA off of microfluidic chips without using enzymes or any deliberate DNA fragmentation protocol. Methods use shear force, physical agitation, or fluid flow to “snap” or “release” the DNA from obstacles or pillars of a capture array. Notably, release of DNA from the device proceeds without the requirement for any deliberate shearing or fragmentation (e.g., no deliberate breaking of a covalent bond within the backbone of the DNA) and from cell lysis to collection of the high-molecular weight DNA (hmwDNA, which is at least a 100 kbp in length for purposes herein), the hmwDNA is always in aqueous solution, and does not need to undergo any drying or phase changes, which could damage DNA. The flow conditions that remove the hmwDNA from the device may accomplish the removal by deforming the device (high pressure bending a wall away from the capture array), by pressure to push the molecules past or through obstacles or pillars; pressure to overcome electrostatic or Van der Waals interactions between DNA and obstacle or pillar surfaces, or to overcome steric resistance, or to disentangle the DNA from itself and straighten & elongate the molecules, etc., or by other mechanisms described herein.

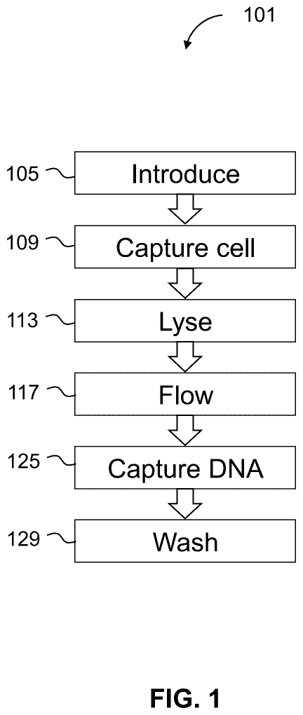

FIG. 1 diagrams steps of a method 101 of extracting or separating nucleic acids from a biological sample. The method 101 includes introducing 105 a sample containing a cell into a channel of a microfluidic device, capturing 109 the cell on one or more cell-capture features disposed within the channel, and lysing 113 the captured cell to release DNA from the cell. Exemplary devices are shown and discussed in greater detail. The lysis step 113 may be performed to lyse or disrupt the membrane of the cell. The lysis process may include one or any combination of reagents, temperature, and mechanical activity. For example, nuclear and cellular membranes may be lysed by heating. Cell and nuclear membrane can also be lysed using mechanical agitation including but not limited to sonication and acoustic waves. In some embodiments, a lysis reagent is flown in through an inlet to the channel The lysis reagent, may include, without limitation, a detergent and a chaotropic salt. In particular, the detergents may be for example, Triton X-100 and/or Tween 20. Chaotropic salts include but are not limited to n-butanol, ethanol, magnesium chloride, sodium dodecyl sulfate. Lysing 113 the cell releases DNA within the channel. The method 101 includes flowing 117 the DNA through the channel to a capture array within the channel and capturing 125 the DNA on the capture array.

The method 101 includes washing 129 the DNA into a collection vessel by flowing a fluid through the channel under conditions that remove the DNA from the capture array thereby collecting, in the collection vessel, at least one DNA molecule of at least one hundred kilobase-pairs in length that was released from the cell.

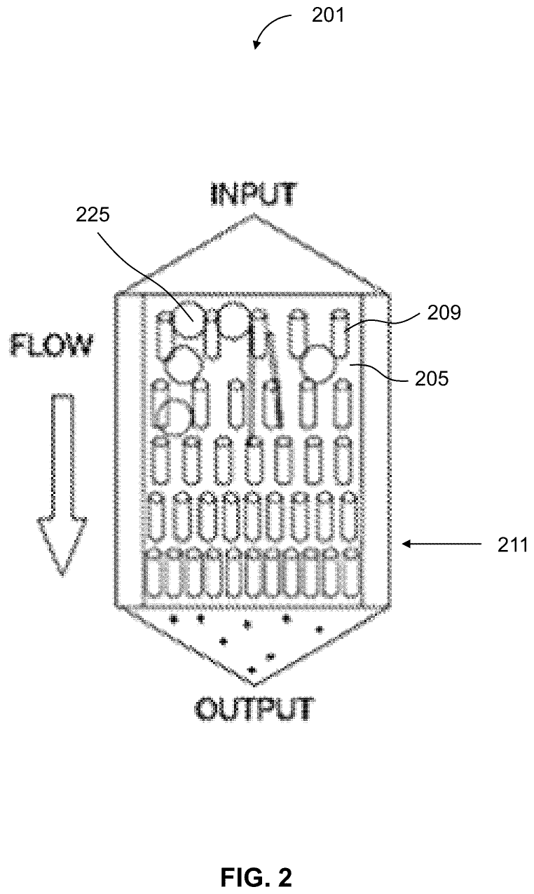

FIG. 2 shows a microfluidic device 201 (e.g., an apparatus for extracting or separating nucleic acids from a biological sample) useful in methods of the invention. The device 201 includes a channel 205 extending from an inlet or input to an outlet or output. As shown, at least one cell 225 has been captured on one or more cell-capture features 209 disposed within a channel 205 while an aqueous liquid is flowing through the channel in the direction indicated by the flow arrow. When the cell 225 is lysed to release genomic DNA, the DNA flows in the direction of flow and is captured 125 on a capture array 211 within the channel.

Using the device 201 in the method 101, the washing step 129 is performed without introducing any restriction enzyme into the microfluidic device 201. The DNA may be (i) maintained in aqueous solution throughout the method, (ii) never exposed to a restriction enzyme or fragmentation protocol, and (iii) collected in a reservoir with a very high molecular weight, e.g., greater than 100 kbp in length. To accomplish those purposes, features and variables of the device 201 and method 101 maybe tuned or adjusted. For example, methods may involve adjusting or controlling features of the obstacles or pillars that constitute the cell-capture features 209 and/or the capture array 211.

In the device 201, the cell-capture features 209 may be provided as micropillars or other such obstacles with diameters between about 2 μm and about 200 μm. Diameters in preferred embodiments are between about 2.5 μm and about 50 μm, between about 15 4 μm and about 20 μm, or between about 5 μm and about 10 μm. The spacing among and between the obstacles or pillars of the call capture features 209 may be about 10 to 20 μm, e.g., about 15 μm. Downstream of the cell capture features 209 is a capture array 211 for nucleic acids.

In the device 201, the capture array 211 may be provided as micropillars or other such obstacles with diameters between about 0.1 μm and about 10 μm, e.g., a few μm, e.g., about 2.

The cell-capture features 209 and the capture array 211 may be discrete and separated portions of the channel 205, or they may be separate functional areas within one array with a (optionally stepped or continual) gradient in size and spacing of obstacles or features. In some embodiments, the cell-capture features 209 and the capture array 211 are provided as an array of microposts (5 μm wide and 20 μm tall), designed with a gradient in spacing to create a solid obstacle for cell capture with downstream region for DNA capture. The average gap between the microposts may vary (stepped or continuously) from 15 μm to 2 μm along the channel. In some embodiments, the relevant dimensions of the microfluidic device 201 included an input channel 205 width of about 50-100 μm, a channel 205 depth of about 20 μm, a cell capture array 209 width of about 200-500 μm, a channel length of about 13 mm, a capture array 211 with a micropillar width of about 4 μm, and a smallest gap between pillars of about 1.5 μm. The microchannels may hold about 50 nL of fluid. In preferred embodiments, the microchannels may hold about 10 μl. The device 201 may include, for capture array 211, nucleic acid entanglement micropillars with a cross-sectional dimension of about 4 μm×4 μm spaced in a gradient that begins with the micropillars being about 10 μm apart and ending with the micropillars being about 7 μm apart. Devices and methods of the invention may use features or techniques discussed in Benitez, 2014, Microfluidic extraction, stretching and analysis of human chromosomal DNA from single cells, Lap Chip 12(22):4848-4854 and/or in Agrawal, 2020, Microfluidic long DNA sample preparation from cells, Lab Chip 19(2):281-290, the contents of both of which are incorporated by reference for all purposes.

To optimize removal of hmwDNA in aqueous solution from the device without using restriction enzymes or a fragmentation protocol one may use array features shown herein including pillar diameter, pillar gap distance, pillar height vs channel height, and pillar density. Methods may include adjusting, setting, or changing flow conditions such as, for example, flow rate, pressure, temperature, and time. One set of features that may be optimized relate to sample density and may include an amount of DNA per pillar, a density of DNA per mm\hat{ }2, and a density of cell loading. For example, some embodiments use the pillar dimensions and spacings described above. An aqueous liquid (de-ionized water, water, saline, a buffer such as a phosphate-buffered solution, etc.) may be flowed through the device 201 at, for example, less than about 100 μL/s for any amount of time, e.g., at least about e.g., 2 minutes Certain embodiments extract and isolate intact, hmwDNA by—after the DNA is captured on the capture array 211—increasing a rate or pressure of flow, e.g., up to at least about 100 μL/s for at least a few seconds.

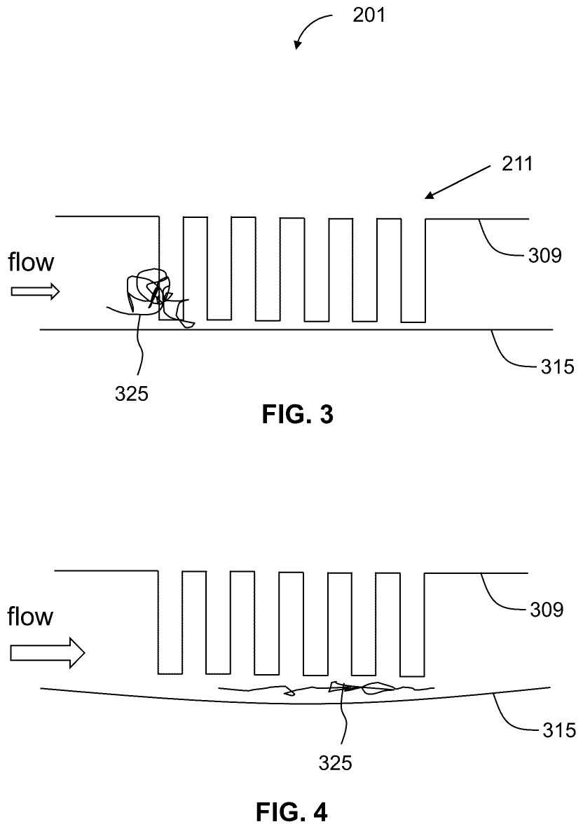

FIG. 3 shows a capture array 211 with an array of pillars or obstacles extending from a first wall 309 of the channel and terminating at ends that sit against (or very close to) a second wall 315, opposed to the first wall. In the image as shown, the pillars point down from above, and the first wall 309 is above the lower, second wall 315. In the figure, the flow arrow is drawn to indicate a direction of flow of an aqueous fluid with a size proportional to flow rate or pressure. As shown, at least one molecule of DNA 325, longer than 100 kbp, is captured on the capture array. To remove the DNA 325 from the device 201, conditions of the flow are changed. The conditions that remove the DNA 325 from the capture array 211 may include an increase in pressure of the fluid flowing through the channel.

FIG. 4 shows a result of increasing pressure of the aqueous liquid flowing through device 201. As shown, the increase in pressure deforms at least a portion of the channel or the capture array to create or increase spacing through which the DNA 325 is removed from the capture array 211. That is, the conditions that remove the DNA 325 from the capture array 211 include an increase in pressure of the fluid that deforms the microfluidic device 201 to move the first wall 309 away from the second wall 315 to introduce or expand a gap between the second wall and the ends of the pillars or obstacles. After the washing step, a collection reservoir collects a plurality of DNA molecules each greater than one hundred kilobase-pairs that were released from the cell by the lysing step.

Any suitable collection vessel or reservoir may be used including, for example, a well or void on the device 201 itself, e.g., in fluid communication with a downstream portion of the channel 205. Certain embodiments use a connected or proximal tube such as a test tube, microcentrifuge tube sold under the trademark EPPENDORF, or a blood collection tube sold under the trademark VACUTAINER, or a conical sample tube sold under the trademark FALCON TUBE. Thus far has been shown a method of extracting or separating high molecular weight DNA from a biological sample. Such a method includes flowing an aqueous fluid containing a cell 225 through a channel 205 of a microfluidic device 201. The cell gets lysed and releases, into aqueous solution in the aqueous fluid, at least one DNA molecule 325 of at least 100 kbp in length. The method includes capturing the DNA molecule 325 on a capture array 211 in the channel 205. Flow conditions of the aqueous fluid are introduced or changed to wash the DNA molecule 325 from the capture array 211 and into a collection reservoir, thereby maintaining the DNA molecule 325 in constant aqueous solution from lysis of the cell 225 to collection in reservoir, thereby providing the at least one DNA molecule 325 of at least one hundred kbp in length in aqueous solution in the collection reservoir.

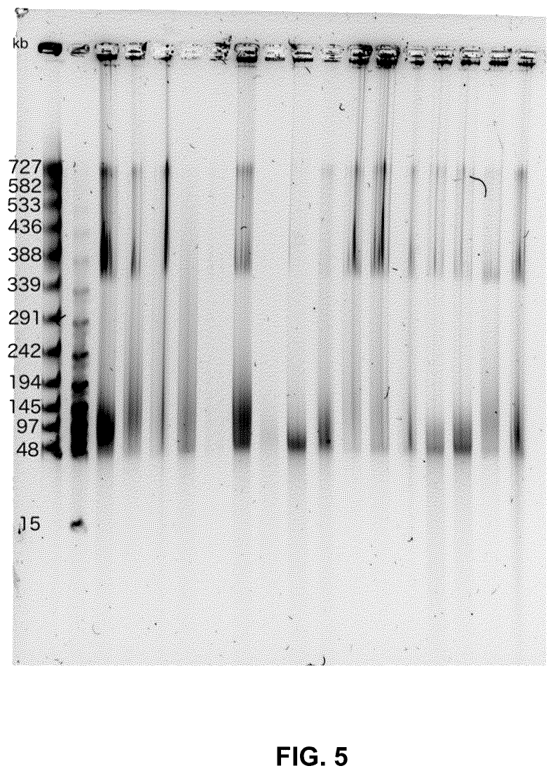

FIG. 5 shows a gel with DNA that was extracted and isolated using methods and apparatuses described herein. Each lane is a product from different runs with minor variations in flow rate, timing, etc. Dark bands appear in almost all of the lanes covering a range of sizes from about 388 kbp to about 727 kbp (including also smaller sizes). While different conditions and mechanism may produce the depicted results, it is theorized that the changing flow conditions include increasing pressure of the aqueous fluid to deform at least a portion of the channel or the capture array to thereby create or increase spacing through which the DNA molecule escapes the capture array. Notably, the DNA being extracted is not exposed to any exogenous endonuclease from lysis of the cell to collection in reservoir. The DNA is in aqueous solution at all times. The DNA collected includes molecules with a range of sizes from about 388 kbp to about 727 kbp.

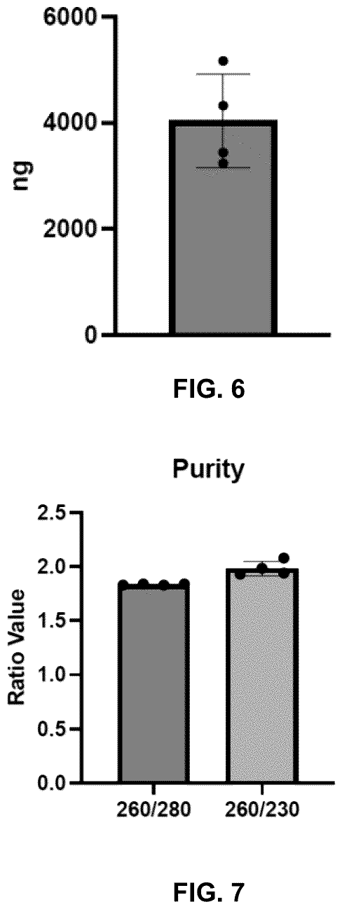

FIG. 6 shows DNA yield in nanograms (ng) from several instrument runs. For the depicted instrument runs, 4 replicate runs were performed. For each run, the input was about 800,000 HeLa cells. There error bars indicate the standard deviation of the measured parameter (applicable also to the purity graph).

FIG. 7 is a bar graph showing purity of DNA collected from the HeLa cells for which yield is shown. The results, including the yield, purity, and gel results, show that methods herein may be used to collect numerous genomic DNA molecules, each at least 500 kbp in length, in a collection reservoir or vessel. After the washing step, the collection vessel collects a plurality of genomic DNA molecules each greater than five hundred kilobase-pairs that were released from the cell by the lysing step. Noting that fluidic coupling of an outlet of the channel 205 to a collection vessel such a microcentrifuge tube does not allow DNA to go anywhere but into the vessel, and it is reasonable to conclude that the genomic DNA molecules in the collection vessel constitute a substantial portion of a genome of the cell, e.g., essentially all except for very minor fragments that may cling within the device.

Other embodiments are within the scope of the disclosure. For example, it may not be necessary that any portion of the device 201 undergo any deformation. It may be that the conditions that remove the DNA from the capture array 211 include an increase in flow rate of the fluid, wherein the increase in the flow rate washes the DNA off of the capture array.

Certain embodiments use magnetic features.

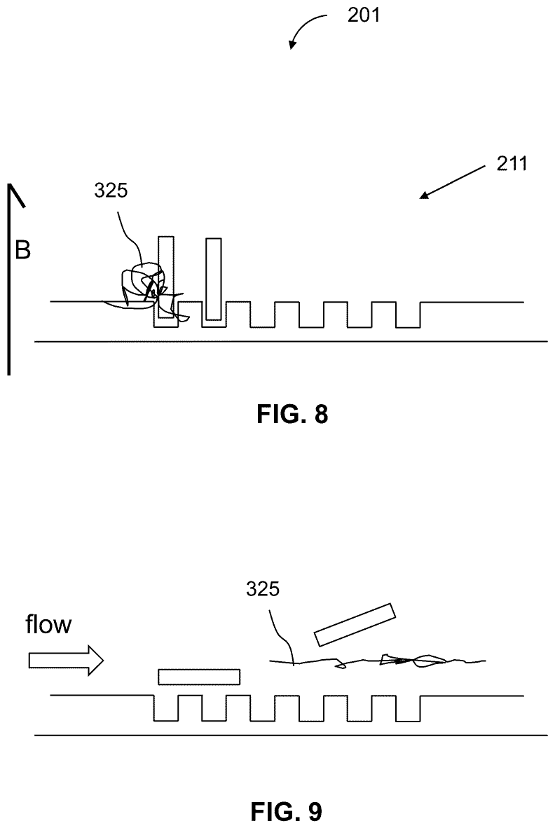

FIG. 8 shows a device in which a capture array 211 includes magnetically-responsive bars biased into a functional position by the presence of a magnetic field B. Here, the conditions that remove the DNA molecule 325 from the capture array 211 may include changing (e.g., removing) the magnetic field B around the microfluidic device to decrease an obstruction presented by the capture array 211.

FIG. 9 shows the device with the magnetically-responsive bars after removal of the field B. As can be seen, the flow of aqueous liquid will wash the DNA molecule 325 out of the channel 205 and into a collection reservoir or vessel. The flow may also wash the magnetically-responsive bars out, but separating those is trivial. As shown, the capture array 211 comprises an array of magnetically responsive structures (which may be bars, rods, beads, pillars, or irregular masses) that change placement or orientation in response to the changing of the magnetic field.

Other embodiments (e.g., “array removal”) may use a capture array 211 comprising an array of solid structures on which the DNA gets captured and wherein the conditions that remove the DNA from the capture array include retracting, dissolving, or melting the solid structures to free the captured DNA. In some embodiments (e.g., “protein pillars”), the capture array 209 comprises an array of proteins on which the DNA gets captured and wherein the conditions that remove the DNA from the capture array include introducing a protease or a reducing agent that degrades the proteins. In certain embodiments (e.g., “open the channel”), the capture array comprises an array of pillars or obstacles extending from a first wall of the channel and the conditions that remove the DNA from the capture array include removing, opening, or lifting away a second wall, opposed to the first all, to allow the fluid to freely wash the DNA out of the microfluidic device. In yet other embodiments (e.g., “salting the DNA off”), the capture array may include an array of obstacles that include a silica resin on which the DNA gets captured and wherein the conditions that remove the DNA from the capture array include washing the capture array with a low salt solution that decreases affinity of the DNA for the silica resin. In certain embodiments (e.g., “magnetic DNA capture beads”), the capture array comprises a plurality of functionalized magnetic DNA capture beads held in place in the channel by a magnetic field, and the conditions that remove the DNA from the capture array include changing or removing magnetic field and washing the DNA capture beads out of the microfluidic device.

Embodiments of devices of the disclosure may have a wide array of cell-capture features that meet a wide array of DNA capture features at a boundary or transition zone. Such devices may be operable to perform the methods and provide the outputs described herein.

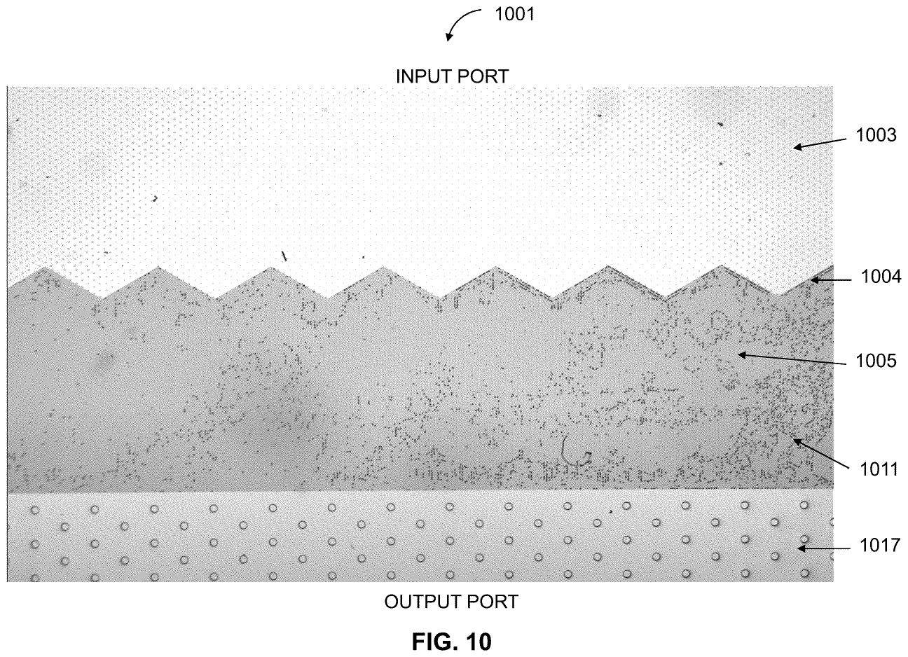

FIG. 10 is a photograph of a device 1001 for the enzyme-free isolation of hmwDNA from one or more cells. The device 1001 has a generally sawtooth shaped boundary or transition zone between a first array of cell capture features 1003 and a second array of microfeatures 1005 that provides a DNA capture array. The device 1001 includes a support 1017 having an inlet port for receiving the sample, an outlet port for dispensing the flow-through, and a microfluidic channel disposed within the support and extending from the inlet port to the outlet port. The microfluidic channel includes a first array of microfeatures 1003 (cell capture) and a second array of microfeatures 1005 (DNA capture).

In the reproduced photomicrograph, the first array of microfeatures 1003 are micropillars that are visible (as dot-like marks in the picture). The second array of microfeatures 1005 includes very fine micropillars that are small enough and close enough together that they appear as a uniform gray color across the middle of the figures. The first array of microfeatures 1003 meets the second array of microfeatures 1005 along a saw-tooth shaped boundary 1004. There is no wall or other structure at the boundary. The boundary 1004 is simply the span across the microchannel at which an aqueous fluid passes from the first array of microfeatures 1003 to the second array of microfeatures 1005.

The device 1001 was manufactured from PDMS and the PDMS included some manufacturing imperfections 1011 that are visible as some irregularly spaced dark marks in the photomicrograph but the imperfections 1011 (dark marks) are not part of any array of microfeatures. The PDMS device 1001 includes a surrounding supporting structure 1017 that appears to include large pillars or columns (visible as about 70 circles in the bottom 10% of the photomicrograph). Those parts of the supporting structure 1017 hold the device 1001 together with appropriate dimensions for sample processing but do not participate directly in sample processing.

This depicted embodiment of the device 1001 shows one apparatus that may be used for extracting or separating hmwDNA (at least 100 kbp) from biological samples, without using any cleavage enzyme or fragmentation protocol, and always keeping the DNA in aqueous solution. The DNA is extracted from a cell by a method that includes (i) flowing an aqueous fluid containing the cell through a channel of the device 1001, wherein the cell gets lysed and releases, into aqueous solution in the aqueous fluid, at least one DNA molecule of at least one hundred kbp in length, (ii) capturing the DNA molecule on a capture array, e.g., second array of microfeatures 1005, in the channel, and (iii) changing flow conditions of the aqueous fluid to wash the DNA from the second array of microfeatures 1005 and out of the output port (a quick increase in flow may blast the DNA off of microfeatures 1005 or expand gaps within the device 1001), thereby maintaining the DNA molecule in constant aqueous solution from when the cell is lysed until DNA is collected a reservoir, and to thus provide the DNA molecule (at least 100 kbp in length) in aqueous solution in the collection reservoir. With device 1001, the purity, yield, and gel results shown herein may be obtained by increasing the flow rate to thus increase the pressure. For example, the flow rate to capture the DNA may be about 100 μL/s and the flow rate may be approximately doubled for at least about 20 seconds to a minute to provide the results shown herein.

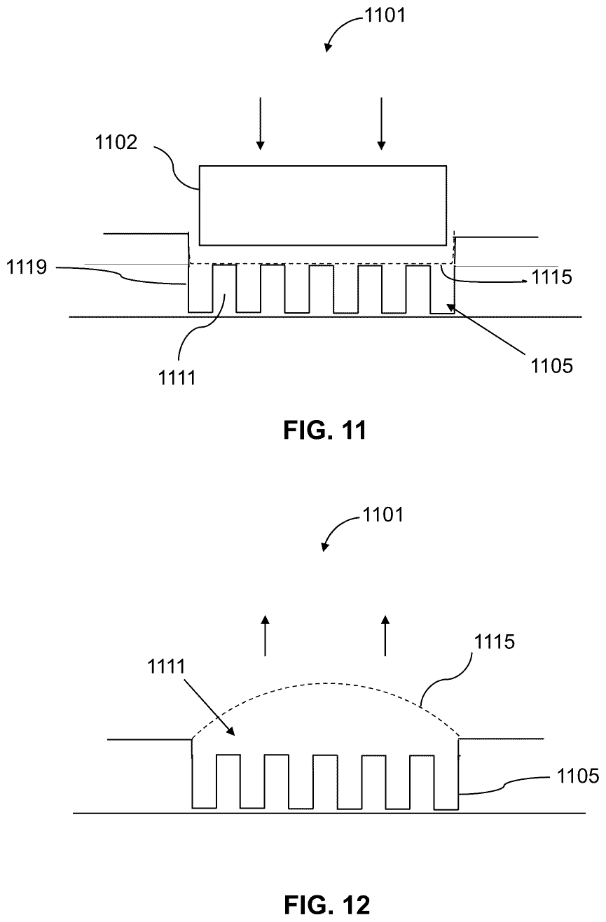

FIG. 11 shows an embodiment of a microfluidic device 1101 with a capture array 1111 in which walls 1119 of a channel 1105 may be taller than the capture array 1111. A film 1115 is positioned near and/or adhered to the channel walls 1109 and a holding member 1102, such as a block or a clamp, is placed on top of the film 1115 to seal the film 1115 to the capture array 1111. The film 1115 may be any flexible plastic or fabric film comprising a material such as TPE or, PDMS, nylon, cling-film, the polyolefin/wax film sold under the trademark PARAFILM, or any other suitable material. The capture array 1111 preferably includes an array of obstacles or pillars. The pillars of the device 1101 may have the same properties as the pillars mentioned throughout the application. In the microfluidic device 1101, the capture array 1111 are pillars that may be contacted or sealed with the film 1115 for performing the method 101. Sealing the film 1115 against the capture array 1111 completes or creates a physical gate or barrier across the channel 1105, promoting successful capture of the DNA on the capture array 1111. When the holding member 1102 sits against the capture array, holding the film 1115 against the capture array 1111, the closure between the film 1115 and the capture array 1111 directs any fluid flowing through the channel to pass through the capture features 1111. That promotes capture of DNA on the capture array 1111.

FIG. 12 shows the microfluidic device 1101 being used in extracting or separating nucleic acids from a biological sample. At the depicted stage, DNA (not shown) has been captured on the capture array 1111, and the DNA is being washed out of the capture array 1111 by flowing a fluid through the channel 1105 under conditions that remove the DNA from the capture array 1111. In the depicted embodiment, the flow conditions of the washing step are obtained by lifting away the holding member 1102 from the capture array 1111. Lifting away the holding member 1102 allows the film 1115 to un-seal from the capture array 1111. The film 1115 can be separated from the capture array 1111 by removing the holding member 1102. As shown, the film 1115 has separated from the capture array 1111. The film 1115 may expand (e.g., stretch, deform, billow, or swell) away from the capture features 1111 and even away from the channel walls 1119, creating a gap or fluidic opening between the capture features 1111 and the film 1115. The gap created by deformation or expansion of the film 1105 permits the DNA to wash off, and away from, the capture array 1111.

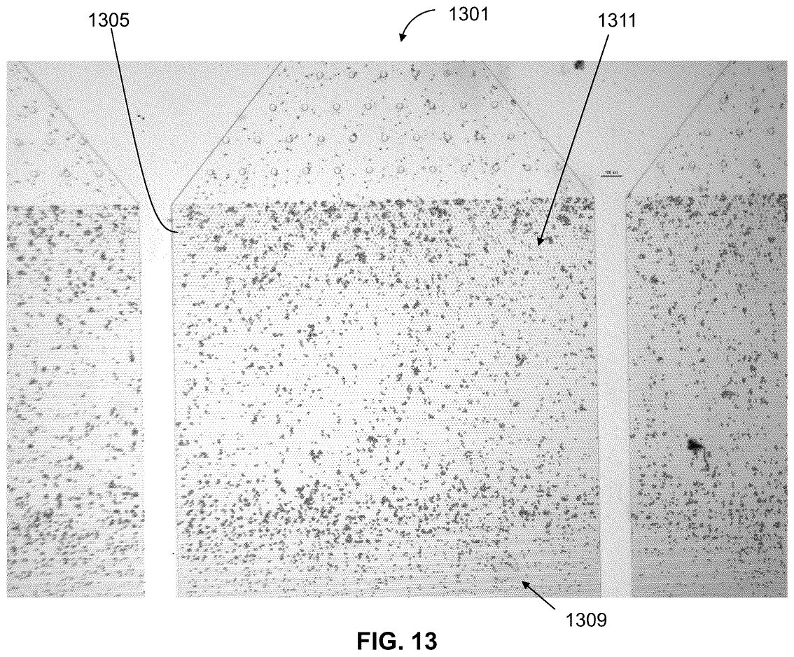

FIG. 13 is a photograph of a microfluidic device 1301 that includes cell-capture features 1309 and a capture array 1311 disposed within a channel 1305. The diameters of the obstacles or pillars of the cell-capture features 1309 and the capture array 1311 are preferably within a range of about 5-10 μm. Spacing, or gaps, between obstacles or pillars of the cell-capture features 1309 and the capture array 1311 preferably vary from about, e.g., 15 μm to about, e.g., 2 μm along the channel (in the image, the spacing is stepped, but the step sizes may be smaller than some viewers will readily discern and the changing spacing may equally be continuous or stepped). Preferably, the depicted channel 1305 has an internal volume on the order of about 10-50 μL. The scale bar is 100 μm. The cell-capture features 1309 and the capture array 1311 were PDMS bonded to glass.

The device 1301 was made and used in performing method 101. A sample with a population of cells was introduced into the channel 1305, and cells were captured on the cell-capture features 1309. The captured cells were lysed to release DNA, which was flowed through the channel 1305 to the capture array 1311. Under flow condition well below about 100 μL/s, e.g., on the order of about 1 to about 10 μL/s, the DNA flowed to, and was captured on, the capture array 1311. Continuous flow elongates the captured DNA allowing the DNA to be analyzed, e.g., fluorescently labeled and visualized by fluorescence microscopy. To remove the DNA from the microfluidic device 1301, the DNA is washed from the capture array by changing flow conditions. Increasing flow rate to a rate on the order of about 100 μL/s was found to remove the DNA from the capture array 1311, allowing the DNA to be collected in a reservoir (here, an off-chip tube). It may be theorized that the increased flow rate induced at least transient deformation in material the device 1301 allowing the DNA to separate from the capture array 1311. The collected DNA included DNA molecules of at least about 700 kilobase-pairs in length, as shown in the gel presented herein.

Embodiments of the disclosure provide library prep methods that provides DNA to which sequencing adaptors have been attached, wherein one or more steps of a library prep method are performed on the device 201. Certain “rapid” library prep methods, referred to as tagmentation approaches, are tagmentation-based and use transposase to cleave DNA on the device 201 and optionally to integrate adapters. Other “ligation” library prep methods, referred to as ligation approaches, include fragmenting DNA and ligating adaptors to the fragments.

In the library prep embodiments, at least one cell is captured at the cell capture structure 209. When the cell 225 is lysed to release genomic DNA, the DNA flows and is captured 125 at the capture site 211 within the channel.

For tagmentation-based methods or approaches, a reagent mix comprising a transposome (e.g., transposase enzyme complexed with transposase adaptors) is delivered to the capture site. The gDNA is mixed with transposase on the device 201. It may be preferable to incubate 30° for about 2 minutes. The transposase cleaves the gDNA and attached transposase adaptors to the ends, at the cleavage sites. In preferred embodiments, reaction of the DNA with the transpose not only attaches the transpose adaptors to the DNA but also fragments the DNA enough to facilitate the easy release of the DNA from the capture site 211. In a subsequent step (discussed in greater detail below), after heating (e.g., 80 degree for about 2 min) sequencing adaptors and buffer may introduced. That mixture may be incubated, e.g., room T for about 5 minutes. In certain embodiments of the tagmentation approach, sequencing adaptors are subsequently attached to transposase-adaptors that are attached to the DNA to yield adaptor-ligated fragments. The sequencing adaptors may be attached to the ends of the transposase adaptors by any suitable methods including, for example, by ligation or annealing. In certain optional embodiments, the transposase adaptors may, themselves, be sequencing adaptors (e.g., Y-adaptors with a double stranded portion and first and second single stranded ends with a motor protein attached the first single stranded portion).

For ligation-based methods or approaches, the DNA is optionally fragmented at the capture site. Any suitable method known in the art may be used to fragment the DNA include digestion with restriction enzymes, sonication, shearing, acid-catalyzed hydrolysis, or combinations thereof. Optionally, DNA is not actively fragmented at the capture site. In a subsequent step, adaptor ligation may attach adaptors to naturally-occurring ends of the DNA such as telomeres and/or adaptor ligation may take advantage of some modest fragmentation (passive fragmentation) or breakage that has occurred during the workflow, such as during lysis rupture and flowing over the pillars. In the subsequent step, sequencing adaptors are ligated to the DNA or the fragments thereof (yielding adaptor-ligated fragments). The adaptor ligation may be performed on the device 201 (e.g., at the capture site 211 or at a second stage downstream of a capture site 211) or off device (in a separate tube or on a fluidically connected second microfluidic chip). Benefits of the ligation based methods are that the on-chip method obviates the need for bead clean-up. Convention protocols exhibit substantial sample loss during cleanup. Here, doing ligation-based methods has shown to greatly improve product yield (e.g., by an integer multiplier). In the ligation approaches, certain embodiments include attaching or ligating adaptors to DNA post-capture at the capture site 211, e.g., while the DNA is captured or entangled on pillars without (intentionally) fragmenting further. While active fragmentation is optional, it is not necessary to actively or further fragment the DNA to ligate. Methods of the disclosure are not limited to ligating adaptors to DNA after an active fragmentation and include any manner of attaching adaptors to DNA even while that DNA is captured at the capture site 211 and without performing any active fragmentation step (“digestion free”). It may be preferable to not introduce further fragmentation (no active fragmentation step) and to attach or ligate adapters to the end of one or more un-fragmented chromosomes or very large (tens of millions of base-pairs) DNA molecule. In the context of methods disclosed herein of recovering chromosome-scale (>hundreds of thousands of bases, preferably at least tens of millions of base pairs, e.g., 50 MBP) intact DNA molecules from the capture site, the described methods allow one to extract or separate very large (e.g., whole chromosome) from a biological sample including from an intact cell.

The very large, e.g., chromosome scale, intact nucleic acid that is extracted or separated from the biological sample containing at least one cell may have an adaptor attached to at least one end. For example, one may obtain a chromosome (or substantial portion thereof) with a sequencing adaptor ligated to at least one end of thereof. The sequencing adaptor may be a Y-adaptor with a motor protein attached to one single stranded end of the Y. As used herein, motor protein comprises one of the proteins with helicase activity such as a phi29 polymerase or bacterial CsgG or modified version thereof available from OXFORD NANOPORE as the R6, R7, R7.3, R9, R9.4, R9.5, R10, or R10.3 motor protein. Those digestion-free ligation approaches may be beneficial because sequencing telomeric regions is challenging. Additionally, digestion-free ligation approaches provide a method by which to sequence an entire chromosome with a sequencing technology such as nanopore sequencing, because such digestion-free ligation approaches allow sequencing to begin at the very end (or beginning) of a chromosome or other template nucleic acid, not somewhere in the middle.

Any suitable sequencing adaptors may be used. For example, the described steps may be used to fragment DNA and attach any of the sequencing adaptors known as Y-adaptors, which have a double-stranded portion that is ligated to a template fragment and two single-stranded ends that do not anneal to each other and also are not available for ligation to another adaptor or fragment.

Depending on the sequencing platform, after ligation to the sequencing adaptors, the sample may be ready for sequencing. In some instances, the ligation may add primer binding sites and preparation for sequencing may involve amplifying the fragments onto beads, optionally with dilution and partitioning in individual reaction volumes (e.g., droplets or wells) with primer-decorated beads (e.g., as used in pyroseqeuncing, IonTorrent sequencing, and Ultima sequencing). In certain embodiments, the addition of Y-adaptors provides a sample that is ready for loading onto a flow cell. In some embodiments, the Y-adaptors are specific for nanopore sequencing. For example, each Y-adaptor may have a motor protein attached to one strand of the single-stranded end of the Y.

By the described means, gDNA or other nucleic acid may be extracted from cellular samples and prepared for sequencing on a device 201. Such a sample preparation apparatus and method supports goals in contemporary genomics of streamlining and automating sample preparation. Specifically, genomics will be made more available by methods and devices that combine extraction, purification, and library prep. Results have shown the proof of concept —that at least a first step of the “rapid” library prep may be performed “on-chip”, on the device 201.

The embodiments combine extraction, purification, and the transposase adapter integration step all in one go. After attachment to transposase adaptors, subsequent steps feature ligation of those transposase adaptors to sequencing adaptors (such as Y-adaptors with a motor protein attached to at least one strand). Ligation to sequencing adaptors may be performed using any suitable hardware including, for example, (i) off-chip in a separate reaction tube; (ii) on-chip in a capture well or volume (a second “stage”) downstream of the capture site 211; or (iii) on-chip on a different chip (e.g., a sequencing flow cell) that is fluidically connected to the device 201. For example, in some embodiments, the device 201 and a sequencing flow cell are both provided as consumables that connect to (e.g., “snap” onto) a laboratory instrument. Fluidic couplings may be included that transfer the transposase adaptor ligated fragments from the capture site 211 into a capture well or volume (the second “stage”), which itself may be on the device 201 or may be on the sequencing flow cell.

Samples have been processed using transposomes and cells on the device 201 as described and sequenced via nanopore sequencing, which validates the potential of on-chip library preparation. Those workflows have included includes washing 129 the transposase-adaptor ligated DNA into a collection vessel by flowing a fluid through the channel under conditions that remove the DNA from the capture array (without the use of restriction enzymes) thereby collecting, in the collection vessel, at least one DNA molecule of at least one hundred kilobase-pairs in length that was released from the cell. It is contemplated that other methods may succeed in washing the library preparation product from the capture site 211 including very gentle washes, stringency/salt manipulations, detergents or electrostatic charge, or removable or meltable micropillars.

It is noted that incubation of gDNA with transposomes here may technically have some effect similar to fragmentation of the DNA, yielding small fragments. However, that is not a primary purpose as described here. Instead, for the described library preparation steps, the transposome may be flowed onto the device 201 at a low concentration such that the primary result is to yield still very long DNA, high-molecular weight (e.g., >100 kilobases) with transposase adaptors attached to the ends.

The sequencing adaptors may be attached on-chip (e.g., in a second “stage”, a reaction pool down a channel for the capture site) or it may be intended to incubate with sequencing adaptors and ligase off-chip (in a fluidically connected downstream chip or after collection into a separate tube such as a microcentrifuge tube).

A washing step may be performed without introducing any restriction enzyme into the microfluidic device 201 to move material away from the capture site 211. Preferably, DNA is (i) maintained in aqueous solution throughout the method, (ii) never exposed to a restriction enzyme or fragmentation protocol aside from the transposase, and (iii) collected in a reservoir with a very high molecular weight, e.g., greater than 100 kbp in length. Features and variables of the device 201 may be any of those described elsewhere herein.

Devices and methods of the invention may use features or techniques discussed in Benitez, 2014, Microfluidic extraction, stretching and analysis of human chromosomal DNA from single cells, Lap Chip 12(22):4848-4854 and/or in Agrawal, 2020, Microfluidic long DNA sample preparation from cells, Lab Chip 19(2):281-290, the contents of both of which are incorporated by reference for all purposes.

1. A method of extracting or separating nucleic acids from a biological sample; the method comprising:
introducing a sample containing a cell into a channel of a microfluidic device;
capturing the cell on one or more cell-capture features disposed within the channel;
lysing the captured cell to release DNA from the cell;
flowing the DNA through the channel to a capture array within the channel;
capturing the DNA on the capture array; and
washing the DNA into a collection reservoir by flowing a fluid through the channel under conditions that remove the DNA from the capture array thereby collecting, in the collection reservoir, at least one DNA molecule at least 100 kilobase-pairs in length that was released from the cell.

2. The method of claim 1, wherein the washing step is performed without introducing any restriction enzyme into the microfluidic device.

3. The method of claim 1, wherein the conditions that remove the DNA from the capture array include an increase in pressure of the fluid flowing through the channel.

4. The method of claim 3, wherein in the increase in pressure deforms at least a portion of the channel or the capture array to create or increase spacing through which the DNA is removed from the capture array.

5. The method of claim 1, wherein the capture array comprises an array of pillars or obstacles extending from a first wall of the channel and terminating at ends that sit against a second wall, opposed to the first wall.

6. The method of claim 5, wherein the conditions that remove the DNA from the capture array include an increase in pressure of the fluid that deforms the microfluidic device to move the first wall away from the second wall to introduce a gap between the second wall and the ends of the pillars or obstacles.

7. The method of claim 1, wherein the conditions that remove the DNA from the capture array include an increase in flow rate of the fluid, wherein the increase in the flow rate washes the DNA off of the capture array.

8. The method of claim 1, wherein the conditions that remove the DNA from the capture array include changing a magnetic field around the microfluidic device to decrease an obstruction presented by the capture array.

9. The method of claim 8, wherein the capture array comprises an array of magnetically responsive structures that change placement or orientation in response to the changing of the magnetic field.

10. The method of claim 1, wherein the capture array comprises one selected from the list consisting of:
(i) an array of solid structures on which the DNA gets captured and wherein the conditions that remove the DNA from the capture array include retracting, dissolving, or melting the solid structures to free the captured DNA;
(ii) an array of proteins on which the DNA gets captured and wherein the conditions that remove the DNA from the capture array include introducing a protease or a reducing agent that degrades the proteins;
(iii) an array of pillars or obstacles extending from a first wall of the channel and wherein the conditions that remove the DNA from the capture array include removing, opening, or lifting away a second wall, opposed to the first all, to allow the fluid to freely wash the DNA out of the microfluidic device;
(iv) an array of obstacles that include a silica resin on which the DNA gets captured and wherein the conditions that remove the DNA from the capture array include washing the capture array with a low salt solution that decreases affinity of the DNA for the silica resin; and
(v) a plurality of functionalized magnetic DNA capture beads held in place in the channel by a magnetic field, and wherein the conditions that remove the DNA from the capture array include changing or removing magnetic field and washing the DNA capture beads out of the microfluidic device.

11. A method of extracting or separating high molecular weight DNA from a biological sample; the method comprising:
flowing an aqueous fluid containing a cell through a channel of a microfluidic device, wherein the cell gets lysed and releases, into aqueous solution in the aqueous fluid, at least one DNA molecule of at least 100 kbp in length;
capturing the DNA molecule on a capture array in the channel; and
changing flow conditions of the aqueous fluid to wash the DNA from the capture array and into a collection reservoir, thereby maintaining the DNA molecule in constant aqueous solution from lysis of the cell to collection in reservoir, thereby providing the at least one DNA molecule of at least 100 kbp in length in aqueous solution in the collection reservoir.

12. The method of claim 11, wherein the changing flow conditions include increasing pressure of the aqueous fluid to deform at least a portion of the channel or the capture array to thereby create or increase spacing through which the DNA molecule escapes the capture array.

13. The method of claim 12, wherein the DNA molecule is not exposed to any exogenous endonuclease from lysis of the cell to collection in reservoir.

14. The method of claim 13, further comprising collecting numerous genomic DNA molecules, each at least 500 kbp in length, in the collection reservoir, wherein the genomic DNA molecules constitute a substantial portion of a genome of the cell.

15. A method of preparing nucleic acid for sequencing; the method comprising:
introducing a sample containing a cell into a channel of a microfluidic device;
capturing the cell at a cell capture structure disposed within the channel;
lysing the captured cell to release DNA;
capturing the DNA at a capture site within the channel; and
flowing a transposome complex to the capture site and, by the action of the transposome, attaching transposase adaptors to segments of the DNA to yield adaptor-linked fragments comprising the transposase-adaptors and the segments.

16. The method of claim 15, further comprising attaching sequencing adaptors to the adaptor-linked fragments.

17. The method of claim 16, wherein the sequencing adaptors are Y-adaptors with a double stranded portion and first and second single stranded ends with a motor protein attached the first single stranded portion.

18. The method of claim 16, wherein the attaching step is performed within a reaction volume on the microfluidic device downstream of the capture site.

19. The method of claim 15, further comprising flowing the adaptor-linked fragments to a sequencing flow cell.

20. The method of claim 15, further comprising flowing library preparation reagents that include the transposome complex to the capture site via a channel that intersects with a DNA flow channel extending from the cell capture structure to the capture site.
